(19) United States
(12) Patent Application Publication
(10) Pub. No.: US 2011/0286946 A1
(43) Pub. Date: Nov. 24, 2011

(54) SUNSCREEN COMPOSITIONS CONTAINING HYDROLYZED JOJOBA ESTERS FOR IMPROVED WATER RESISTANCE

(75) Inventors: Tiffany Oliphant, Chandler, AZ (US); David A. Ashley, Phoenix, AZ (US)

(73) Assignee: International Flora Technologies Ltd., Chandler, AZ (US)

(21) Appl. No.: 13/114,658

(22) Filed: May 24, 2011

Related U.S. Application Data
(60) Provisional application No. 61/347,740, filed on May 24, 2010.

(51) Int. Cl. A61K 8/37; A61Q 17/04

(52) U.S. Cl. 424/60; 424/59

(57) ABSTRACT

Sunscreen compositions suitable for topical application are provided. The sunscreen composition includes at least one sunscreen active. Further, the sunscreen composition contains a water resistance agent including hydrolyzed jojoba esters, jojoba esters, and water. The water resistance agent provides for trapping the sunscreen active against the user's skin so that the sunscreen active is not removed by exposure to water. The sunscreen composition may further include potassium jojobate and/or jojoba alcohols. Also, the sunscreen composition may include glycerin for skin hydration purposes.

### PRIORITY CLAIM

This application claims priority to U.S. Provisional Application Ser. No. 61/347,740 entitled “Hydrolyzed Jojoba Ester Sunscreen Fixative” and filed on May 24, 2010. That application is incorporated herein by reference in its entirety.

### TECHNICAL FIELD

This document generally relates to sunscreen compositions, and more particularly relates to sunscreen compositions with improved water resistance.

### BACKGROUND

Sunscreen compositions applied to the skin provide protection against the damaging effects of ultraviolet radiation. In recent years, general knowledge regarding the damaging effects to skin from the sun's ultraviolet radiation has grown. As a result, use of sunscreen compositions has become the norm for people who spend time in sunlight.

Sunscreen compositions include at least one active sunscreen agent, which is typically an ultraviolet light-absorbing ingredient. Because active sunscreen agents generally absorb ultraviolet light in a limited range of wavelengths, sunscreen compositions often utilize multiple active sunscreen agents which offer absorption of different or partially overlapping ranges of wavelengths. In order to effectively offer protection from ultraviolet radiation, sunscreen agents must have good substantivity, i.e., a tendency to resist being easily removed or to persist on the skin. For example, it is important that a sunscreen composition be resistant to removal via normal rubbing or brushing against clothes, furniture, or other normal contact experienced by a person's skin. Further, because many consumers use sunscreen compositions in connection with water sports, on hot days, and/or in connection with strenuous physical activities, it is important that a sunscreen composition have water resistance so that it is not lost due to contact with water or perspiration. Further, water resistance improves substantivity.

While improved substantivity is a well-known goal, there exist other considerations for sunscreen compositions. Importantly, sunscreen compositions must not irritate the skin or have any longer term health impacts. Further, sunscreen compositions should be easy to apply to the skin, as well as easy to clean up after application. At the same time, sunscreen compositions should not stain fabrics during normal use. Also, the sunscreen composition's texture, scent, and appearance must be pleasing.

While the cosmetic industry continues its ongoing effort to identify a method of forming a sufficiently water resistant sunscreen that meets the consumers' other requirements, there remains a need for sunscreen compositions having improved substantivity, especially in terms of water resistance.

Accordingly, it is desirable to provide a sunscreen composition for topical application having improved water resistance. Furthermore, other desirable features and characteristics of the present invention will become apparent from the subsequent detailed description of the invention and the appended claims, taken in conjunction with this background of the invention.

### BRIEF SUMMARY

Sunscreen compositions suitable for topical application are provided. In accordance with one embodiment, a sunscreen composition includes at least one sunscreen active. Further, the sunscreen composition contains a water resistance agent including hydrolyzed jojoba esters, jojoba esters, and water. In certain embodiments, the water resistance agent forms 0.2% of the sunscreen composition. Functionally, the water resistance agent provides for trapping the sunscreen active against the user's skin so that the sunscreen active is not removed by exposure to water, or by exposure to water followed by physical contact. The sunscreen composition may further include potassium jojobate and/or jojoba alcohols. Also, the sunscreen composition may include glycerin for skin hydration purposes.

In another embodiment, the sunscreen composition suitable for topical application comprises at least one sunscreen active and hydrolyzed jojoba esters. Such an embodiment may further include jojoba esters and/or water. The embodiment may also include potassium jojobate or jojoba alcohols. Further, it may comprise glycerin.

Also provided is an embodiment of a water-resistant sunscreen composition suitable for topical application. In this embodiment, at least one sunscreen active is provided. Also provided is a water resistance agent comprising hydrolyzed jojoba esters and water. In an exemplary embodiment, the hydrolyzed jojoba esters form about 16 wt. % of the sunscreen composition. The sunscreen active or actives may form about 12 wt. % of the sunscreen composition.

In each of the embodiments, the sunscreen active may be one or more of the following compounds, one or more salts of the following compounds, and/or one or more derivatives of the following compounds: acetaminosalol; allantoin PABA; 4-aminobenzoic acid; 4-aminobenzoic acid; benzalphthalide; benzophenone-1; benzophenone-2; benzophenone-3; benzophenone-4; benzophenone-5; benzophenone-6; benzophenone-7; benzophenone-8; benzophenone-9; benzophenone-10; benzophenone-11; benzophenone-12; benzotriazolyl dodecyl p-cresol; 3-benzylidene camphor; benzylidenecamphor hydrolyzed collagen sulfonamide; benzylidene camphor sulfonic acid; benzyl salicylate; bisdisulizole disodium; bis(butylbenzoate)diaminotriazine; aminopropyltrisiloxane; bis-ethylhexyloxyphenyl triazine; bornelone; bumetrizole; butyl methoxydibenzoylmethane; calcium cerium oxide; calophyllum inophyllum seed oil; camellia sinensis leaf extract; camphor benzalkonium methosulfate; carotenoids; cerium (3+) carbonate, calcination products with silica; cerium (3+) carbonate, calcination products with silica and talc; cinoxate; cobalt dna; DEA-methoxycinnamate; deschampsia Antarctica leaf extract; diacetylcurcumin; dibenzoxazoyl naphthalene; di-t-butyl hydroxybenzylidene camphor; diethylamino methoxycinnamate; diacetylcurcumin; diethylamino hydroxybenzoyl hexyl benzoate; diethylhexyl butamido triazone; diethylhexyl 2,6-naphthalate; digalloyl trioleate; diisopropylmethyl cinnamate; dimethicone/peg-15 crosspolymer; 1-(3,4-dimethoxyphenyl)-4,4-dimethyl-1,3-pentanedione; di-methoxycinnamidopropyl ethyldimoniuim chloride ether; 4-(dimethylamino)-,3-methylbutyl benzoate; dimethyl PABA ethylcetearyl dimomium tosylate; dimorpholinopyridazinone; diphenyl carbomethoxy acetoxy naphthopyran; diphenylmethyl piperazinylbenzimidazole; disodium bisethylphenyl triaminotriazone; stilbenedisulfonate; disodium distyrylbiphenyl disulfonate; disodium phenyl dibenzimidazole tetrasulfonate; di-t-butyl hydroxybenzylidene camphor; drometrizole; drometrizole trisiloxane; ethyl cinnamate; ethyl dihydroxypropyl PABA; ethyl diisopropyl cinnamate; ethylhexyl bis-isopentylbenzoxazblylphenyl melamine; ethylhexyl dimethoxybenzylidene dioxoimidazolidine propionate; ethylhexyl dimethyl PABA; ethylhexyl ferulate; ethylhexyl methoxycinnamate; 2-ethylhexyl 4-(N-methyl-N-nitrosamino)benzoate; ethylhexyl salicylate; ethylhexyl triazone; ethyl methoxycinnamate; ethylhexyl methoxycrylene; ethylhexyl methoxydibenzoylmethane; ethyl trimethylbenzoyl phenylphosphinate; etocrylene; fluorescent brightener 230; 4-(2-β-glucopyranosiloxy)propoxy-2hydroxybenzophenone; glyceryl ethylhexanoate dimethoxycinnamate; glyceryl ethyklhexanoate dimethoxycinnamate; glyceryl ethylhexanoate dimethoxycinnamate; glyceryl ethylhexanoate dimethoxycinnamate; glyceryl PABA; glycol salicylate; gossypium herbaceum (cotton) seed extract; helianthus annuus (sunflower) seed extract; hexanediol disalicylate; hexyloxy trimethylphenol; homosalate; hydrolyzed aspergillus/ginseng extract ferment; hydrolyzed euglena gracilis extract; hydrolyzed linseed extract; hydrolyzed olive fruit; hydrolyzed wheat bran; hydrolyzed soy extract; 2-hydroxy-1,4-naphthalenedione; hydroxypropyl phenylhydrazinoyl t-butylcarbamate; indocyanine green; isoamyl p-methoxycinnamate; isobutyl phenylhydrazinoyl methanesulfonamide; isopropylbenzyl salicylate; isopropyl dibenzoylmethane; isopropylmethoxy cinnamate; lawsone; limonia acidissima bark extract; limonia acidissima bark powder; Madura aurantica wood extract; maxafil; methoxycinnamidopropyl C18-22 alkyldimonium tosylate; methoxycinnamidopropyl hydroxysultaine; methoxycinnamidopropyl laurdimonium tosylate; methoxycinnamidopropyl polysilsesquioxane; methoxycinnamidopropyl silsesquioxane silicate; methyl acrylate/methylene drometrizole; methacrylate copolymer; menthyl anthranilate; menthyl salicylate; 4-methylbenzylidene camphor; 4-methylbenzylidene camphor sulfonic acid; 5-methykl-2-phenylbenzoxazole; methylene bis-benzotriazolyl tetramethylbutylphenol; momordica cochinchinensis seed aril oil; octocrylene; octrizole; octyl dimethyl 4-aminobenzoic acid; PEG-25 PABA; pentyl 4-(dimethylamino) benzoate; phenylbenzimidazole sulfonic acid; pinus pinaster bark/bud extract; polyacrylamidomethyl benzylidene camphor; polysilicone-15; potassium methoxycinnamate; prinsepia utilis seed extract; red petrolatum; rutinyl succinate; scytonemin; spirulina patensis powder; TEA-salicylate; terephthalylidene dicamphor sulfonic acid; tetrahydrocurcumin diacetate; titanium dioxide; triPABA panthenol; tris-biphenykl triazine; vitis vinifera (grape) seed extract; and zinc cerium oxide.

### DETAILED DESCRIPTION

The following detailed description of the invention is merely exemplary in nature and is not intended to limit the invention or the application and uses of the invention. Furthermore, there is no intention to be bound by any theory presented in the preceding background of the invention or the following detailed description of the invention.

Sunscreen compositions for topical application and having increased water resistance are provided herein. In order to provide increased water resistance, the sunscreen compositions include hydrolyzed jojoba esters. The hydrolyzation of unsaponifiables such as those found in jojoba oil is discussed in U.S. Pat. No. 7,435,424, issued on Oct. 14, 2008 and assigned to International Flora Technologies, Ltd., and incorporated herein by reference.

Generally, hydrolyzed jojoba esters are produced by the reaction of aqueous alkali metal hydroxides, e.g., KOH (the preferred hydroxide), NaOH, LiOH, CaOH, MgOH, and the like, with refined jojoba oil. Refined jojoba oil contains various proportions of long chain diunsaturated esters. Hydrolysates of refined jojoba oil are nearly a 55:45 mixture of polar hydrophilic long chain salts (alkali salts) and relatively non-polar lipophilic materials (fatty alcohols). The lipophilic fraction is unsaponifiable material, i.e., material that remains water insoluble after the completion of a saponification reaction. (As defined by A.O.C.S. Official Method Ca 6b-53, unsaponifiable materials are those substances frequently found as components of fats and oils, which cannot be saponified by the usual caustic treatment, but which are soluble in ordinary fats and oils).

Typically, the carbon chain lengths of both the hydrophilic and lipophilic hydrolysates include and vary from C₁₈ to C₂₄ and have ω-9 double bonds as part of each molecule. It has been determined by the inventors that the combination of saponifiable and unsaponifiable fractions of the jojoba hydrolysates has properties that aid in the formulation of sunscreen compositions. Specifically, hydrolyzed jojoba esters can contain and impart useful properties to applied surfaces. These surfaces may be animate surfaces, particularly human skin, plant surfaces, and even the surfaces of inanimate objects, for example, objects of wood, fiber, or plastic. In particular, hydrolyzed jojoba esters can improve substantivity and water resistance on human skin.

In a first exemplary embodiment, the hydrolyzed jojoba esters are used in conjunction with other ingredients to form a water resistance agent. Specifically, the water resistance agent includes the hydrolyzed jojoba esters, jojoba esters, and water. In this exemplary embodiment, the water resistance agent may form 0.2% or 0.5% of the sunscreen composition. As required by its purpose, the sunscreen composition will also include at least one sunscreen active that absorbs ultraviolet radiation. Further, the sunscreen composition may include jojoba oil, potassium jojobate and/or jojoba alcohols. The sunscreen composition can include glycerin for skin hydration. Typically, the sunscreen composition may also include other thickening, dispersing, suspending and emulsifying agents; chelating agents; pH neutralizing agents; preservatives; humectants; emollients; lubricants; and fragrances.

The water resistance agent is believed to work by trapping the sunscreen active against the skin of the user. As a result, the water resistance agent prevents exposure of the sunscreen active to water. Generally, exposure to water shortens the substantivity of sunscreen actives. Therefore, by forming a barrier between the sunscreen active and the water, the water resistance agent is able to increase the duration of ultraviolet absorption by the sunscreen active.

In this embodiment, the water resistance agent will exhibit a specific gravity at 25° C. of between about 0.85 and about 1.0, and a flash point (Cleveland open cup) above 290° C. With solubility defined as forming one phase at 1:1 at 25° C., it is noted that the exemplary water resistance agent is soluble in ethyl alcohol, isopropyl jojobate, isopropyl alcohol, oleyl alcohol, and propylene glycol; partially soluble in polysorbate 20 and polysorbate 80; and insoluble in acetone, butylene glycol, caprylic/capric triglyceride, castor oil, cyclomethicone, decyl glucoside, decyl oleate, dimethicone, ethyl acetate, glycerine, isononyl isonoanoate, isopropyl myristate, isopropyl palmitate, jojoba oil, lanolin oil, mineral oil, octyldodecyl stearoyl stearate, PPG-15 stearyl ether, sodium lauryl sulfate (30% solution), sorbitol, sunflower oil, and water.

Testing of this first exemplary embodiment was performed with a base sunscreen comprising the following active agents: 0.5% benzophenone-3; 1.9% octyl methoxycinnamate; and 0.75% octyl salicylate. Specifically, the base sunscreen was tested as Article A; the base sunscreen plus 0.5% of the water resistance agent of this first exemplary embodiment was tested as Article B; the base sunscreen plus 0.2% of the water resistance agent of this first exemplary embodiment was tested as Article C; and the base sunscreen with 2% Dermacryl 79 (an industry standard sunscreen waterproof ingredient, product of Indopco, Inc. DBA National Starch and Chemical Company, Bridgewater, N.J.) was tested as Article D.

On Day One of testing, three sites having an area of approximately one-inch by one-inch were demarcated on each volar forearm of testing subjects. One site on each forearm was untreated. The other two sites on each forearm were treated with either 40 μl of A; 12.5% base sunscreen in ethanol; 25% base sunscreen in ethanol; or 50% base sunscreen in alcohol. For each test article, 40 μl was dispensed and evenly rubbed onto the designated skin area. After treatment, each site was allowed to air dry for fifteen minutes. ATR-FTIR (attenuated total reflectance—Fourier transform infrared spectroscopy) measurements were then taken at each treatment site to measure the infrared spectrum of the skin.

On Day Two of testing, two sites having an area of approximately one-inch by one-inch were demarcated on each volar forearm, with no overlap with previous sites. An ATR-FTIR measurement was taken of the untreated site. A test article was then applied to one designated area on each forearm, and allowed to air dry for fifteen minutes.

Then, each arm was immersed in separate containers of turbulent, room-temperature fresh water for twenty minutes, immediately followed by air drying for twenty minutes. This immersion and drying cycle was repeated until a total immersion time of eighty minutes was reached. After the last immersion, each forearm was air dried with warm air from a blow dryer. ATR-FTIR measurements were then taken.

On Day Three, the Day Two process was repeated, though salt water approximating the concentration of sweat (sodium chloride, 1147 mg/liter) was used instead of fresh water.

The following tables show the results of testing:

TABLE I
Percent Retention Octyl Salicylate - Fresh Water

| | Article A | Article B | Article C | Article D |
|---|---|---|---|---|
| Mean Area Under Peak | 0.0149 | 0.0122 | 0.0151 | 0.0111 |
| Mean Area Under Peak | 0.0149 | 0.0122 | 0.0151 | 0.0111 |
| (excluding outliers) | | | | |
| Concentration OS | 2.5896 | 2.2329 | 2.6143 | 2.0884 |
| Remaining (mg/cm2) | | | | |
| Percent Increase | N/A | −13.8% | 1.0% | −19.4% |
| (relative to vehicle) | | | | |
| Concentration OS | 35.4154 | 35.4154 | 35.4154 | 35.4154 |
| Applied (mg/cm2) | | | | |
| Percent OS Retained | 7.1% | 6.1% | 7.2% | 5.7% |

TABLE II
Percent Retention Octyl Salicylate - Salt Water

| | Article A | Article B | Article C | Article D |
|---|---|---|---|---|
| Mean Area Under Peak | 0.0135 | 0.0128 | −0.0502 | 0.0102 |
| Mean Area Under Peak | 0.0135 | 0.0128 | 0.0144 | 0.0102 |
| (excluding outliers) | | | | |
| Concentration OS | 2.4050 | 2.3068 | 2.5138 | 1.9724 |
| Remaining (mg/cm2) | | | | |
| Percent Increase | N/A | −4.1% | 4.5% | −18.0% |
| (relative to vehicle) | | | | |
| Concentration OS | 36.4154 | 36.4154 | 36.4154 | 36.4154 |
| Applied (mg/cm2) | | | | |
| Percent OS Retained | 6.6% | 6.3% | 6.9% | 5.4% |

TABLE III
Percent Retention Benzophenone-3 - Fresh Water

| | Article A | Article B | Article C | Article D |
|---|---|---|---|---|
| Mean Area Under Peak | 0.0003 | 0.0006 | −0.0005 | 0.0007 |
| Mean Area Under Peak | 0.0003 | 0.0006 | 0.00001 | −0.0006 |
| (excluding outliers) | | | | |
| Concentration B-3 | 7.3081 | 10.4849 | 8.3628 | 6.2036 |
| Remaining (mg/cm2) | | | | |
| Percent Increase | N/A | 43.5% | 14.4% | −15.1% |
| (relative to vehicle) | | | | |
| Concentration B-3 | 24.2769 | 24.2769 | 24.2769 | 24.2769 |
| Applied (mg/cm2) | | | | |
| Percent B-3 Retained | 30.1% | 43.2% | 34.4% | 25.6% |

TABLE IV
Percent Retention Benzophenone-3 - Salt Water

| | Article A | Article B | Article C | Article D |
|---|---|---|---|---|
| Mean Area Under Peak | −0.0006 | 0.0008 | 0.0389 | −0.0003 |
| Mean Area Under Peak | −0.0006 | 0.0008 | 0.0023 | −0.0003 |
| (excluding outliers) | | | | |
| Concentration B-3 | 6.4596 | 10.8465 | 15.9809 | 7.2550 |
| Remaining (mg/cm2) | | | | |
| Percent Increase | N/A | 67.9% | 147.4% | 12.3% |
| (relative to vehicle) | | | | |
| Concentration B-3 | 24.2769 | 24.2769 | 24.2769 | 24.2769 |
| Applied (mg/cm2) | | | | |
| Percent B-3 Retained | 26.6% | 44.7% | 65.8% | 29.9% |

TABLE V
Percent Retention Octyl Methoxycinnamate - Salt Water

| | Article A | Article B | Article C | Article D |
|---|---|---|---|---|
| Mean Area Under Peak | 0.0597 | 0.0377 | 0.0581 | 0.0257 |
| Mean Area Under Peak | 0.0597 | 0.0377 | 0.0581 | 0.0257 |
| (excluding outliers) | | | | |
| Concentration OMC | 78.0396 | 4.5576 | 72.6883 | −35.3556 |
| Remaining (mg/cm2) | | | | |
| Percent Increase | N/A | −94.2% | −6.9% | −145.3% |
| (relative to vehicle) | | | | |
| Concentration OMC | 92.2523 | 92.2523 | 92.2523 | 92.2523 |
| Applied (mg/cm2) | | | | |
| Percent OMC Retained | 84.6% | 4.9% | 78.8% | −38.3% |

TABLE VI
Percent Retention Octyl Methoxycinnamate - Salt Water

| | Article A | Article B | Article C | Article D |
|---|---|---|---|---|
| Mean Area Under Peak | 0.0544 | 0.0437 | 0.0166 | 0.0334 |
| Mean Area Under Peak | 0.0544 | 0.0437 | 0.0594 | 0.0334 |
| (excluding outliers) | | | | |
| Concentration OMC | 60.4249 | 24.6000 | 76.8780 | −9.7126 |
| Remaining (mg/cm2) | | | | |
| Percent Increase | N/A | −59.3% | 27.2% | −116.1% |
| (relative to vehicle) | | | | |
| Concentration OMC | 92.2523 | 92.2523 | 92.2523 | 92.2523 |
| Applied (mg/cm2) | | | | |
| Percent OMC Retained | 65.5% | 26.7% | 83.3% | −10.5% |

As shown in Table I, Article C exhibited slightly higher octyl salicylate (OS) retention than the test vehicle (Article A). In Table II, Article C showed slightly higher octyl salicylate retention (0.3%) than the vehicle. In Table III, Article B and Article C retained more Benzophenone-3 (B-3) than the test vehicle. Table IV shows that Article B, Article C, and Article D all exhibited increased retention of Benzophenone-3 over the test vehicle under exposure to salt water. In Table V, the vehicle showed the greatest retention of octyl methoxycinnamate (OMC) upon exposure to fresh water. However, Table VI, shows that, upon exposure to salt water, Article C retained 17.8% more octyl methoxycinnamate than the vehicle.

As is clear from the testing results, the base sunscreen plus 0.2% of the water resistance agent of this first exemplary embodiment produced the greatest retention of each of the three sunscreen actives under salt water conditions. Further, it was the only test article to produce retention of octyl salicylate in both salt and fresh water conditions. Also, both test articles containing the water resistance agent of the first exemplary embodiment (Articles B and C) always showed greater retention of the sunscreen actives than the industry standard (Article D).

In a second embodiment, hydrolyzed jojoba esters are utilized without other jojoba-derived components. For example, the water resistance agent in the sunscreen composition may comprise only hydrolyzed jojoba esters and water. Such a water resistance agent can have a water content of 80% and a hydrolyzed jojoba esters content of 20%. Further, the exemplary water resistance agent will exhibit a specific gravity at 25° C. of between about 0.85 and about 1.0, and a flash point (Cleveland open cup) above 290° C. With solubility defined as forming one phase at 1:1 at 25° C., it is noted that the exemplary water resistance agent is soluble in ethyl alcohol, glycerine, isopropyl alcohol, and polysorbate 20; partially soluble in water, acetone, decyl glucoside, isopropyl jojobate, propylene glycol, sodium lauryl sulfate (30% solution); and insoluble in butylene glycol, caprylic/capric triglyceride, castor oil, cyclomethicone, decyl oleate, dimethicone, ethyl acetate, isononyl isonoanoate, isopropyl myristate, isopropyl palmitate, jojoba oil, lanolin oil, mineral oil, octyldodecyl stearoyl stearate, oleyl alcohol, polysorbate 80, PPG-15 stearyl ether, sorbitol, and sunflower oil.

Testing of this second exemplary embodiment was also performed with a base sunscreen comprising the following active agents: 0.5% benzophenone-3; 1.9% octyl methoxycinnamate; and 0.75% octyl salicylate. Specifically, the base sunscreen was tested as Article A; the base sunscreen plus 1% of the water resistance agent of the second exemplary embodiment was tested as Article B; and the base sunscreen with 2% Dermacryl 79 was tested as Article C.

Testing followed the same procedure as stated above. The following tables illustrate the results of testing:

TABLE VII
Percent Retention Octyl Salicylate - Fresh Water

| | Article A | Article B | Article C |
|---|---|---|---|
| Mean Area Under Peak | 0.0149 | 0.0101 | 0.0111 |
| Mean Area Under Peak | 0.0149 | 0.0106 | 0.0111 |
| (excluding outliers) | | | |
| Concentration OS | 2.5896 | 2.0291 | 2.0884 |
| Remaining (mg/cm2) | | | |
| Percent Increase | N/A | −21.6% | −19.4% |
| (relative to vehicle) | | | |
| Concentration OS | 35.4154 | 35.4154 | 35.4154 |
| Applied (mg/cm2) | | | |
| Percent OS Retained | 7.1% | 5.6% | 5.7% |

TABLE VIII
Percent Retention Octyl Salicylate - Salt Water

| | Article A | Article B | Article C |
|---|---|---|---|
| Mean Area Under Peak | 0.0135 | 0.1132 | 0.0102 |
| Mean Area Under Peak | 0.0135 | 0.1132 | 0.0102 |
| (excluding outliers) | | | |
| Concentration OS | 2.4050 | 2.1198 | 1.9724 |
| Remaining (mg/cm2) | | | |
| Percent Increase | N/A | −11.9% | −18.0% |
| (relative to vehicle) | | | |
| Concentration OS | 36.4154 | 36.4154 | 36.4154 |
| Applied (mg/cm2) | | | |
| Percent OS Retained | 6.6% | 5.8% | 5.4% |

TABLE IX
Percent Retention Benzophenone-3 - Fresh Water

| | Article A | Article B | Article C |
|---|---|---|---|
| Mean Area Under Peak | 0.0003 | 0.0004 | 0.0007 |
| Mean Area Under Peak | 0.0003 | 0.0004 | −0.0006 |
| (excluding outliers) | | | |
| Concentration B-3 | 7.3081 | 9.64601 | 6.2036 |
| Remaining (mg/cm2) | | | |
| Percent Increase | N/A | 31.9% | −15.1% |
| (relative to vehicle) | | | |
| Concentration B-3 | 24.2769 | 24.2769 | 24.2769 |
| Applied (mg/cm2) | | | |
| Percent B-3 Retained | 30.1% | 39.7% | 25.6% |

TABLE X
Percent Retention Benzophenone-3 - Salt Water

| | Article A | Article B | Article C |
|---|---|---|---|
| Mean Area Under Peak | −0.0006 | 0.0005 | −0.0003 |
| Mean Area Under Peak | −0.0006 | 0.0014 | −0.0003 |
| (excluding outliers) | | | |
| Concentration B-3 | 6.4596 | 12.8550 | 7.2550 |
| Remaining (mg/cm2) | | | |
| Percent Increase | N/A | 99.0% | 12.3% |
| (relative to vehicle) | | | |
| Concentration B-3 | 24.2769 | 24.2769 | 24.2769 |
| Applied (mg/cm2) | | | |
| Percent B-3 Retained | 26.6% | 53.0% | 29.9% |

TABLE XI
Percent Retention Octyl Methoxycinnamate - Fresh Water

| | Article A | Article B | Article C |
|---|---|---|---|
| Mean Area Under Peak | 0.0597 | 0.0287 | 0.0257 |
| Mean Area Under Peak | 0.0597 | 0.0392 | 0.0257 |
| (excluding outliers) | | | |
| Concentration OMC | 78.0396 | −14.5443 | −35.3556 |
| Remaining (mg/cm2) | | | |
| Percent Increase | N/A | −118.6% | −145.3% |
| (relative to vehicle) | | | |
| Concentration OMC | 92.2523 | 92.2523 | 92.2523 |
| Applied (mg/cm2) | | | |
| Percent OMC Retained | 84.6% | −15.8% | −38.3% |

TABLE XII
Percent Retention Octyl Methoxycinnamate - Salt Water

| | Article A | Article B | Article C |
|---|---|---|---|
| Mean Area Under Peak | 0.0544 | 0.0323 | 0.0334 |
| Mean Area Under Peak | 0.0544 | 0.0373 | 0.0334 |
| (excluding outliers) | | | |
| Concentration OMC | 60.4249 | 30.3534 | −9.7126 |
| Remaining (mg/cm2) | | | |
| Percent Increase | N/A | −94.5% | −116.1% |
| (relative to vehicle) | | | |
| Concentration OMC | 92.2523 | 92.2523 | 92.2523 |
| Applied (mg/cm2) | | | |
| Percent OMC Retained | 65.5% | 3.6% | −10.5% |

As can be seen from Tables VII-XII, Article B (the base sunscreen plus 1% of the water resistance agent of the second exemplary embodiment) performed substantially the same as the industry standard (Article C) in retaining octyl salicylate in fresh water conditions, and outperformed the industry standard in all other tests.

In a third exemplary embodiment, the sunscreen composition may include the following constituents along with other constituents: 46.65 wt. % deionized water; 16.67 wt. % hydrolyzed jojoba esters, 3.33 wt. % jojoba oil, and 12.0 wt. % sunscreen actives.

While a single sunscreen active may be utilized, more commonly more than one will be used. For instance, the third exemplary embodiment may use 7.00 wt. % ethylhexyl methoxycinnamate, 3.00 wt. % ethylhexyl salicylate, and 2.00 wt. % benzophenone-3.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A sunscreen composition suitable for topical application comprising:
at least one sunscreen active; and
a water resistance agent including hydrolyzed jojoba esters, jojoba esters, and water.

2. The sunscreen composition of claim 1 wherein the water resistance agent forms 0.2% of the sunscreen composition.

3. The sunscreen composition of claim 1 further comprising potassium jojobate.

4. The sunscreen composition of claim 3 further comprising jojoba alcohols.

5. The sunscreen composition of claim 4 further comprising glycerin for skin hydration.

6. The sunscreen composition of claim 1 wherein the at least one sunscreen active is selected from the group consisting of acetaminosalol; allantoin PABA; 4-aminobenzoic acid; 4-aminobenzoic acid; benzalphthalide; benzophenone-1; benzophenone-2; benzophenone-3; benzophenone-4; benzophenone-5; benzophenone-6; benzophenone-7; benzophenone-8; benzophenone-9; benzophenone-10; benzophenone-11; benzophenone-12; benzotriazolyl dodecyl p-cresol; 3-benzylidene camphor; benzylidenecamphor hydrolyzed collagen sulfonamide; benzylidene camphor sulfonic acid; benzyl salicylate; bisdisulizole disodium; bis(butylbenzoate); aminopropyltrisiloxane; bis-ethylhexyloxyphenyl triazine; bornelone; bumetrizole; butyl methoxydibenzoylmethane; calcium cerium oxide; calophyllum inophyllum seed oil; camellia sinensis leaf extract; camphor benzalkonium methosulfate; carotenoids; cerium (3+) carbonate, calcination products with silica; cerium (3+) carbonate, calcination products with silica and talc; cinoxate; cobalt dna; DEA-methoxycinnamate; deschampsia Antarctica leaf extract; diacetylcurcumin; dibenzoxazoyl naphthalene; di-t-butyl hydroxybenzylidene camphor; diethylamino methoxycinnamate; diacetylcurcumin; diethylamino hydroxybenzoyl hexyl benzoate; diethylhexyl butamido triazone; diethylhexyl 2,6-naphthalate; digalloyl trioleate; diisopropylmethyl cinnamate; dimethicone/peg-15 crosspolymer; 1-(3,4-dimethoxyphenyl)-4,4-dimethyl-1,3-pentanedione; di-methoxycinnamidopropyl ethyldimoniuim chloride ether; 4-(dimethylamino)-,3-methylbutyl benzoate; dimethyl PABA ethylcetearyl dimomium tosylate; dimorpholinopyridazinone; diphenyl carbomethoxy acetoxy naphthopyran; diphenylmethyl piperazinylbenzimidazole; disodium bisethylphenyl triaminotriazone; stilbenedisulfonate; disodium distyrylbiphenyl disulfonate; disodium phenyl dibenzimidazole tetrasulfonate; di-t-butyl hydroxybenzylidene camphor; drometrizole; drometrizole trisiloxane; ethyl cinnamate; ethyl dihydroxypropyl PABA; ethyl diisopropyl cinnamate; ethylhexyl bis-isopentylbenzoxazblylphenyl melamine; ethylhexyl dimethoxybenzylidene dioxoimidazolidine propionate; ethylhexyl dimethyl PABA; ethylhexyl ferulate; ethylhexyl methoxycinnamate; 2-ethylhexyl 4-(N-methyl-N-nitrosamino) benzoate; ethylhexyl salicylate; ethylhexyl triazone; ethyl methoxycinnamate; ethylhexyl methoxycrylene; ethylhexyl methoxydibenzoylmethane; ethyl trimethylbenzoyl phenylphosphinate; etocrylene; fluorescent brightener 230; 4-(2-β-glucopyranosiloxy)propoxy-2hydroxybenzophenone; glyceryl ethylhexanoate dimethoxycinnamate; glyceryl ethyklhexanoate dimethoxycinnamate; glyceryl ethylhexanoate dimethoxycinnamate; glyceryl ethylhexanoate dimethoxycinnamate; glyceryl PABA; glycol salicylate; gossypium herbaceum (cotton) seed extract; helianthus annuus (sunflower) seed extract; hexanediol disalicylate; hexyloxy trimethylphenol; homosalate; hydrolyzed aspergillus/ginseng extract ferment; hydrolyzed euglena gracilis extract; hydrolyzed linseed extract; hydrolyzed olive fruit; hydrolyzed wheat bran; hydrolyzed soy extract; 2-hydroxy-1,4-naphthalenedione; hydroxypropyl phenylhydrazinoyl t-butylcarbamate; indocyanine green; isoamyl p-methoxycinnamate; isobutyl phenylhydrazinoyl methanesulfonamide; isopropylbenzyl salicylate; isopropyl dibenzoylmethane; isopropylmethoxy cinnamate; lawsone; limonia acidissima bark extract; limonia acidissima bark powder; Madura aurantica wood extract; maxafil; methoxycinnamidopropyl C18-22 alkyldimonium tosylate; methoxycinnamidopropyl hydroxysultaine; methoxycinnamidopropyl laurdimonium tosylate; methoxycinnamidopropyl polysilsesquioxane; methoxycinnamidopropyl silsesquioxane silicate; methyl acrylate/methylene drometrizole; methacrylate copolymer; menthyl anthranilate; menthyl salicylate; 4-methylbenzylidene camphor; 4-methylbenzylidene camphor sulfonic acid; 5-methykl-2-phenylbenzoxazole; methylene bis-benzotriazolyl tetramethylbutylphenol; momordica cochinchinensis seed aril oil; octocrylene; octrizole; octyl dimethyl 4-aminobenzoic acid; PEG-25 PABA; pentyl 4-(dimethylamino) benzoate; phenylbenzimidazole sulfonic acid; pinus pinaster bark/bud extract; polyacrylamidomethyl benzylidene camphor; polysilicone-15; potassium methoxycinnamate; prinsepia utilis seed extract; red petrolatum; rutinyl succinate; scytonemin; spirulina patensis powder; TEA-salicylate; terephthalylidene dicamphor sulfonic acid; tetrahydrocurcumin diacetate; titanium dioxide; triPABA panthenol; tris-biphenykl triazine; vitis vinifera (grape) seed extract; and zinc cerium oxide.

7. The sunscreen composition of claim 1 wherein the at least one sunscreen active is a salt of a compound selected from the group consisting of acetaminosalol; allantoin PABA; 4-aminobenzoic acid; 4-aminobenzoic acid; benzalphthalide; benzophenone-1; benzophenone-2; benzophenone-3; benzophenone-4; benzophenone-5; benzophenone-6; benzophenone-7; benzophenone-8; benzophenone-9; benzophenone-10; benzophenone-11; benzophenone-12; benzotriazolyl dodecyl p-cresol; 3-benzylidene camphor; benzylidenecamphor hydrolyzed collagen sulfonamide; benzylidene camphor sulfonic acid; benzyl salicylate; bisdisulizole disodium; bis(butylbenzoate) diaminotriazine; aminopropyltrisiloxane; bis-ethylhexyloxyphenyl triazine; bornelone; bumetrizole; butyl methoxydibenzoylmethane; calcium cerium oxide; calophyllum inophyllum seed oil; camellia sinensis leaf extract; camphor benzalkonium methosulfate; carotenoids; cerium (3+) carbonate, calcination products with silica; cerium (3+) carbonate, calcination products with silica and talc; cinoxate; cobalt dna; DEA-methoxycinnamate; deschampsia Antarctica leaf extract; diacetylcurcumin; dibenzoxazoyl naphthalene; di-t-butyl hydroxybenzylidene camphor; diethylamino methoxycinnamate; diacetylcurcumin; diethylamino hydroxybenzoyl hexyl benzoate; diethylhexyl butamido triazone; diethylhexyl 2,6-naphthalate; digalloyl trioleate; diisopropylmethyl cinnamate; dimethicone/peg-15 crosspolymer; 1-(3,4-dimethoxyphenyl)-4,4-dimethyl-1,3-pentanedione; di-methoxycinnamidopropyl ethyldimoniuim chloride ether; 4-(dimethylamino)-,3-methylbutyl benzoate; dimethyl PABA ethylcetearyl dimomium tosylate; dimorpholinopyridazinone; diphenyl carbomethoxy acetoxy naphthopyran; diphenylmethyl piperazinylbenzimidazole; disodium bisethylphenyl triaminotriazone; stilbenedisulfonate; disodium distyrylbiphenyl disulfonate; disodium phenyl dibenzimidazole tetrasulfonate; di-t-butyl hydroxybenzylidene camphor; drometrizole; drometrizole trisiloxane; ethyl cinnamate; ethyl dihydroxypropyl PABA; ethyl diisopropyl cinnamate; ethylhexyl bis-isopentylbenzoxazblylphenyl melamine; ethylhexyl dimethoxybenzylidene dioxoimidazolidine propionate; ethylhexyl dimethyl PABA; ethylhexyl ferulate; ethylhexyl methoxycinnamate; 2-ethylhexyl 4-(N-methyl-N-nitrosamino) benzoate; ethylhexyl salicylate; ethylhexyl triazone; ethyl methoxycinnamate; ethylhexyl methoxycrylene; ethylhexyl methoxydibenzoylmethane; ethyl trimethylbenzoyl phenylphosphinate; etocrylene; fluorescent brightener 230; 4-(2-β-glucopyranosiloxy) propoxy-2hydroxybenzophenone; glyceryl ethylhexanoate dimethoxycinnamate; glyceryl ethyklhexanoate dimethoxycinnamate; glyceryl ethylhexanoate dimethoxycinnamate; glyceryl ethylhexanoate dimethoxycinnamate; glyceryl PABA; glycol salicylate; gossypium herbaceum (cotton) seed extract; helianthus annuus (sunflower) seed extract; hexanediol disalicylate; hexyloxy trimethylphenol; homosalate; hydrolyzed aspergillus/ginseng extract ferment; hydrolyzed euglena gracilis extract; hydrolyzed linseed extract; hydrolyzed olive fruit; hydrolyzed wheat bran; hydrolyzed soy extract; 2-hydroxy-1,4-naphthalenedione; hydroxypropyl phenylhydrazinoyl t-butylcarbamate; indocyanine green; isoamyl p-methoxycinnamate; isobutyl phenylhydrazinoyl methanesulfonamide; isopropylbenzyl salicylate; isopropyl dibenzoylmethane; isopropylmethoxy cinnamate; lawsone; limonia acidissima bark extract; limonia acidissima bark powder; Madura aurantica wood extract; maxafil; methoxycinnamidopropyl C18-22 alkyldimonium tosylate; methoxycinnamidopropyl hydroxysultaine; methoxycinnamidopropyl laurdimonium tosylate; methoxycinnamidopropyl polysilsesquioxane; methoxycinnamidopropyl silsesquioxane silicate; methyl acrylate/methylene drometrizole; methacrylate copolymer; menthyl anthranilate; menthyl salicylate; 4-methylbenzylidene camphor; 4-methylbenzylidene camphor sulfonic acid; 5-methykl-2-phenylbenzoxazole; methylene bis-benzotriazolyl tetramethylbutylphenol; momordica cochinchinensis seed aril oil; octocrylene; octrizole; octyl dimethyl 4-aminobenzoic acid; PEG-25 PABA; pentyl 4-(dimethylamino) benzoate; phenylbenzimidazole sulfonic acid; pinus pinaster bark/bud extract; polyacrylamidomethyl benzylidene camphor; polysilicone-15; potassium methoxycinnamate; prinsepia utilis seed extract; red petrolatum; rutinyl succinate; scytonemin; spirulina patensis powder; TEA-salicylate; terephthalylidene dicamphor sulfonic acid; tetrahydrocurcumin diacetate; titanium dioxide; triPABA panthenol; tris-biphenykl triazine; vitis vinifera (grape) seed extract; and zinc cerium oxide.

8. The sunscreen composition of claim 1 wherein the at least one sunscreen active is a derivative of a compound selected from the group consisting of acetaminosalol; allantoin PABA; 4-aminobenzoic acid; 4-aminobenzoic acid; benzalphthalide; benzophenone-1; benzophenone-2; benzophenone-3; benzophenone-4; benzophenone-5; benzophenone-6; benzophenone-7; benzophenone-8; benzophenone-9; benzophenone-10; benzophenone-11; benzophenone-12; benzotriazolyl dodecyl p-cresol; 3-benzylidene camphor; benzylidenecamphor hydrolyzed collagen sulfonamide; benzylidene camphor sulfonic acid; benzyl salicylate; bisdisulizole disodium; bis(butylbenzoate)diaminotriazine; aminopropyltrisiloxane; bis-ethylhexyloxyphenyl triazine; bornelone; bumetrizole; butyl methoxydibenzoylmethane; calcium cerium oxide; calophyllum inophyllum seed oil; camellia sinensis leaf extract; camphor benzalkonium methosulfate; carotenoids; cerium (3+) carbonate, calcination products with silica; cerium (3+) carbonate, calcination products with silica and talc; cinoxate; cobalt dna; DEA-methoxycinnamate; deschampsia Antarctica leaf extract; diacetylcurcumin; dibenzoxazoyl naphthalene; di-t-butyl hydroxybenzylidene camphor; diethylamino methoxycinnamate; diacetylcurcumin; diethylamino hydroxybenzoyl hexyl benzoate; diethylhexyl butamido triazone; diethylhexyl 2,6-naphthalate; digalloyl trioleate; diisopropylmethyl cinnamate; dimethicone/peg-15 crosspolymer; 1-(3,4-dimethoxyphenyl)-4,4-dimethyl-1,3-pentanedione; di-methoxycinnamidopropyl ethyldimoniuim chloride ether; 4-(dimethylamino)-,3-methylbutyl benzoate; dimethyl PABA ethylcetearyl dimomium tosylate; dimorpholinopyridazinone; diphenyl carbomethoxy acetoxy naphthopyran; diphenylmethyl piperazinylbenzimidazole; disodium bisethylphenyl triaminotriazone; stilbenedisulfonate; disodium distyrylbiphenyl disulfonate; disodium phenyl dibenzimidazole tetrasulfonate; di-t-butyl hydroxybenzylidene camphor; drometrizole; drometrizole trisiloxane; ethyl cinnamate; ethyl dihydroxypropyl PABA; ethyl diisopropyl cinnamate; ethylhexyl bis-isopentylbenzoxazblylphenyl melamine; ethylhexyl dimethoxybenzylidene dioxoimidazolidine propionate; ethylhexyl dimethyl PABA; ethylhexyl ferulate; ethylhexyl methoxycinnamate; 2-ethylhexyl 4-(N-methyl-N-nitrosamino)benzoate; ethylhexyl salicylate; ethylhexyl triazone; ethyl methoxycinnamate; ethylhexyl methoxycrylene; ethylhexyl methoxydibenzoylmethane; ethyl trimethylbenzoyl phenylphosphinate; etocrylene; fluorescent brightener 230; 4-(2-β-glucopyranosiloxy)propoxy-2hydroxybenzophenone; glyceryl ethylhexanoate dimethoxycinnamate; glyceryl ethyklhexanoate dimethoxycinnamate; glyceryl ethylhexanoate dimethoxycinnamate; glyceryl ethylhexanoate dimethoxycinnamate; glyceryl PABA; glycol salicylate; gossypium herbaceum (cotton) seed extract; helianthus annuus (sunflower) seed extract; hexanediol disalicylate; hexyloxy trimethylphenol; homosalate; hydrolyzed aspergillus/ginseng extract ferment; hydrolyzed euglena gracilis extract; hydrolyzed linseed extract; hydrolyzed olive fruit; hydrolyzed wheat bran; hydrolyzed soy extract; 2-hydroxy-1,4-naphthalenedione; hydroxypropyl phenylhydrazinoyl t-butylcarbamate; indocyanine green; isoamyl p-methoxycinnamate; isobutyl phenylhydrazinoyl methanesulfonamide; isopropylbenzyl salicylate; isopropyl dibenzoylmethane; isopropylmethoxy cinnamate; lawsone; limonia acidissima bark extract; limonia acidissima bark powder; Madura aurantica wood extract; maxafil; methoxycinnamidopropyl C18-22 alkyldimonium tosylate; methoxycinnamidopropyl hydroxysultaine; methoxycinnamidopropyl laurdimonium tosylate; methoxycinnamidopropyl polysilsesquioxane; methoxycinnamidopropyl silsesquioxane silicate; methyl acrylate/methylene drometrizole; methacrylate copolymer; menthyl anthranilate; menthyl salicylate; 4-methylbenzylidene camphor; 4-methylbenzylidene camphor sulfonic acid; 5-methykl-2-phenylbenzoxazole; methylene bis-benzotriazolyl tetramethylbutylphenol; momordica cochinchinensis seed aril oil; octocrylene; octrizole; octyl dimethyl 4-aminobenzoic acid; PEG-25 PABA; pentyl 4-(dimethylamino)benzoate; phenylbenzimidazole sulfonic acid; pinus pinaster bark/bud extract; polyacrylamidomethyl benzylidene camphor; polysilicone-15; potassium methoxycinnamate; prinsepia utilis seed extract; red petrolatum; rutinyl succinate; scytonemin; spirulina patensis powder; TEA-salicylate; terephthalylidene dicamphor sulfonic acid; tetrahydrocurcumin diacetate; titanium dioxide; triPABA panthenol; tris-biphenykl triazine; vitis vinifera (grape) seed extract; and zinc cerium oxide.

9. A sunscreen composition suitable for topical application comprising:
at least one sunscreen active; and
hydrolyzed jojoba esters.

10. The sunscreen composition of claim 9 further comprising jojoba esters.

11. The sunscreen composition of claim 10 further comprising water.

12. The sunscreen composition of claim 11 further comprising potassium jojobate.

13. The sunscreen composition of claim 12 further comprising jojoba alcohols.

14. The sunscreen composition of claim 13 further comprising glycerin.

15. The sunscreen composition of claim 9 wherein the at least one sunscreen active is selected from the group consisting of acetaminosalol; allantoin PABA; 4-aminobenzoic acid; 4-aminobenzoic acid; benzalphthalide; benzophenone-1; benzophenone-2; benzophenone-3; benzophenone-4; benzophenone-5; benzophenone-6; benzophenone-7; benzophenone-8; benzophenone-9; benzophenone-10; benzophenone-11; benzophenone-12; benzotriazolyl dodecyl p-cresol; 3-benzylidene camphor; benzylidenecamphor hydrolyzed collagen sulfonamide; benzylidene camphor sulfonic acid; benzyl salicylate; bisdisulizole disodium; bis(butylbenzoate) diaminotriazine; aminopropyltrisiloxane; bis-ethylhexyloxyphenyl triazine; bornelone; bumetrizole; butyl methoxydibenzoylmethane; calcium cerium oxide; calophyllum inophyllum seed oil; camellia sinensis leaf extract; camphor benzalkonium methosulfate; carotenoids; cerium (3+) carbonate, calcination products with silica; cerium (3+) carbonate, calcination products with silica and talc; cinoxate; cobalt dna; DEA-methoxycinnamate; deschampsia Antarctica leaf extract; diacetylcurcumin; dibenzoxazoyl naphthalene; di-t-butyl hydroxybenzylidene camphor; diethylamino methoxycinnamate; diacetylcurcumin; diethylamino hydroxybenzoyl hexyl benzoate; diethylhexyl butamido triazone; diethylhexyl 2,6-naphthalate; digalloyl trioleate; diisopropylmethyl cinnamate; dimethicone/peg-15 crosspolymer; 1-(3,4-dimethoxyphenyl)-4,4-dimethyl-1,3-pentanedione; di-methoxycinnamidopropyl ethyldimoniuim chloride ether; 4-(dimethylamino)-,3-methylbutyl benzoate; dimethyl PABA ethylcetearyl dimomium tosylate; dimorpholinopyridazinone; diphenyl carbomethoxy acetoxy naphthopyran; diphenylmethyl piperazinylbenzimidazole; disodium bisethylphenyl triaminotriazone; stilbenedisulfonate; disodium distyrylbiphenyl disulfonate; disodium phenyl dibenzimidazole tetrasulfonate; di-t-butyl hydroxybenzylidene camphor; drometrizole; drometrizole trisiloxane; ethyl cinnamate; ethyl dihydroxypropyl PABA; ethyl diisopropyl cinnamate; ethylhexyl bis-isopentylbenzoxazblylphenyl melamine; ethylhexyl dimethoxybenzylidene dioxoimidazolidine propionate; ethylhexyl dimethyl PABA; ethylhexyl ferulate; ethylhexyl methoxycinnamate; 2-ethylhexyl 4-(N-methyl-N-nitrosamino)benzoate; ethylhexyl salicylate; ethylhexyl triazone; ethyl methoxycinnamate; ethylhexyl methoxycrylene; ethylhexyl methoxydibenzoylmethane; ethyl trimethylbenzoyl phenylphosphinate; etocrylene; fluorescent brightener 230; 4-(2-β-glucopyranosiloxy)propoxy-2hydroxybenzophenone; glyceryl ethylhexanoate dimethoxycinnamate; glyceryl ethyklhexanoate dimethoxycinnamate; glyceryl ethylhexanoate dimethoxycinnamate; glyceryl ethylhexanoate dimethoxycinnamate; glyceryl PABA; glycol salicylate; gossypium herbaceum (cotton) seed extract; helianthus annuus (sunflower) seed extract; hexanediol disalicylate; hexyloxy trimethylphenol; homosalate; hydrolyzed aspergillus/ginseng extract ferment; hydrolyzed euglena gracilis extract; hydrolyzed linseed extract; hydrolyzed olive fruit; hydrolyzed wheat bran; hydrolyzed soy extract; 2-hydroxy-1,4-naphthalenedione; hydroxypropyl phenylhydrazinoyl t-butylcarbamate; indocyanine green; isoamyl p-methoxycinnamate; isobutyl phenylhydrazinoyl methanesulfonamide; isopropylbenzyl salicylate; isopropyl dibenzoylmethane; isopropylmethoxy cinnamate; lawsone; limonia acidissima bark extract; limonia acidissima bark powder; Madura aurantica wood extract; maxafil; methoxycinnamidopropyl C18-22 alkyldimonium tosylate; methoxycinnamidopropyl hydroxysultaine; methoxycinnamidopropyl laurdimonium tosylate; methoxycinnamidopropyl polysilsesquioxane; methoxycinnamidopropyl silsesquioxane silicate; methyl acrylate/methylene drometrizole; methacrylate copolymer; menthyl anthranilate; menthyl salicylate; 4-methylbenzylidene camphor; 4-methylbenzylidene camphor sulfonic acid; 5-methykl-2-phenylbenzoxazole; methylene bis-benzotriazolyl tetramethylbutylphenol; momordica cochinchinensis seed aril oil; octocrylene; octrizole; octyl dimethyl 4-aminobenzoic acid; PEG-25 PABA; pentyl 4-(dimethylamino)benzoate; phenylbenzimidazole sulfonic acid; pinus pinaster bark/bud extract; polyacrylamidomethyl benzylidene camphor; polysilicone-15; potassium methoxycinnamate; prinsepia utilis seed extract; red petrolatum; rutinyl succinate; scytonemin; spirulina patensis powder; TEA-salicylate; terephthalylidene dicamphor sulfonic acid; tetrahydrocurcumin diacetate; titanium dioxide; triPABA panthenol; tris-biphenykl triazine; vitis vinifera (grape) seed extract; and zinc cerium oxide.

16. The sunscreen composition of claim 9 wherein the at least one sunscreen active is a salt of a compound selected from the group consisting of acetaminosalol; allantoin PABA; 4-aminobenzoic acid; 4-aminobenzoic acid; benzalphthalide; benzophenone-1; benzophenone-2; benzophenone-3; benzophenone-4; benzophenone-5; benzophenone-6; benzophenone-7; benzophenone-8; benzophenone-9; benzophenone-10; benzophenone-11; benzophenone-12; benzotriazolyl dodecyl p-cresol; 3-benzylidene camphor; benzylidenecamphor hydrolyzed collagen sulfonamide; benzylidene camphor sulfonic acid; benzyl salicylate; bisdisulizole disodium; bis(butylbenzoate) diaminotriazine; aminopropyltrisiloxane; bis-ethylhexyloxyphenyl triazine; bornelone; bumetrizole; butyl methoxydibenzoylmethane; calcium cerium oxide; calophyllum inophyllum seed oil; camellia sinensis leaf extract; camphor benzalkonium methosulfate; carotenoids; cerium (3+) carbonate, calcination products with silica; cerium (3+) carbonate, calcination products with silica and talc; cinoxate; cobalt dna; DEA-methoxycinnamate; deschampsia Antarctica leaf extract; diacetylcurcumin; dibenzoxazoyl naphthalene; di-t-butyl hydroxybenzylidene camphor; diethylamino methoxycinnamate; diacetylcurcumin; diethylamino hydroxybenzoyl hexyl benzoate; diethylhexyl butamido triazone; diethylhexyl 2,6-naphthalate; digalloyl trioleate; diisopropylmethyl cinnamate; dimethicone/peg-15 crosspolymer; 1-(3,4-dimethoxyphenyl)-4,4-dimethyl-1,3-pentanedione; di-methoxycinnamidopropyl ethyldimoniuim chloride ether; 4-(dimethylamino)-,3-methylbutyl benzoate; dimethyl PABA ethylcetearyl dimomium tosylate; dimorpholinopyridazinone; diphenyl carbomethoxy acetoxy naphthopyran; diphenylmethyl piperazinylbenzimidazole; disodium bisethylphenyl triaminotriazone; stilbenedisulfonate; disodium distyrylbiphenyl disulfonate; disodium phenyl dibenzimidazole tetrasulfonate; di-t-butyl hydroxybenzylidene camphor; drometrizole; drometrizole trisiloxane; ethyl cinnamate; ethyl dihydroxypropyl PABA; ethyl diisopropyl cinnamate; ethylhexyl bis-isopentylbenzoxazblylphenyl melamine; ethylhexyl dimethoxybenzylidene dioxoimidazolidine propionate; ethylhexyl dimethyl PABA; ethylhexyl ferulate; ethylhexyl methoxycinnamate; 2-ethylhexyl 4-(N-methyl-N-nitrosamino)benzoate; ethylhexyl salicylate; ethylhexyl triazone; ethyl methoxycinnamate; ethylhexyl methoxycrylene; ethylhexyl methoxydibenzoylmethane; ethyl trimethylbenzoyl phenylphosphinate; etocrylene; fluorescent brightener 230; 4-(2-β-glucopyranosiloxy)propoxy-2hydroxybenzophenone; glyceryl ethylhexanoate dimethoxycinnamate; glyceryl ethyklhexanoate dimethoxycinnamate; glyceryl ethylhexanoate dimethoxycinnamate; glyceryl ethylhexanoate dimethoxycinnamate; glyceryl PABA; glycol salicylate; gossypium herbaceum (cotton) seed extract; helianthus annuus (sunflower) seed extract; hexanediol disalicylate; hexyloxy trimethylphenol; homosalate; hydrolyzed aspergillus/ginseng extract ferment; hydrolyzed euglena gracilis extract; hydrolyzed linseed extract; hydrolyzed olive fruit; hydrolyzed wheat bran; hydrolyzed soy extract; 2-hydroxy-1,4-naphthalenedione; hydroxypropyl phenylhydrazinoyl t-butylcarbamate; indocyanine green; isoamyl p-methoxycinnamate; isobutyl phenylhydrazinoyl methanesulfonamide; isopropylbenzyl salicylate; isopropyl dibenzoylmethane; isopropylmethoxy cinnamate; lawsone; limonia acidissima bark extract; limonia acidissima bark powder; Madura aurantica wood extract; maxafil; methoxycinnamidopropyl C18-22 alkyldimonium tosylate; methoxycinnamidopropyl hydroxysultaine; methoxycinnamidopropyl laurdimonium tosylate; methoxycinnamidopropyl polysilsesquioxane; methoxycinnamidopropyl silsesquioxane silicate; methyl acrylate/methylene drometrizole; methacrylate copolymer; menthyl anthranilate; menthyl salicylate; 4-methylbenzylidene camphor; 4-methylbenzylidene camphor sulfonic acid; 5-methykl-2-phenylbenzoxazole; methylene bis-benzotriazolyl tetramethylbutylphenol; momordica cochinchinensis seed aril oil; octocrylene; octrizole; octyl dimethyl 4-aminobenzoic acid; PEG-25 PABA; pentyl 4-(dimethylamino)benzoate; phenylbenzimidazole sulfonic acid; pinus pinaster bark/bud extract; polyacrylamidomethyl benzylidene camphor; polysilicone-15; potassium methoxycinnamate; prinsepia utilis seed extract; red petrolatum; rutinyl succinate; scytonemin; spirulina patensis powder; TEA-salicylate; terephthalylidene dicamphor sulfonic acid; tetrahydrocurcumin diacetate; titanium dioxide; triPABA panthenol; tris-biphenykl triazine; vitis vinifera (grape) seed extract; and zinc cerium oxide.

17. The sunscreen composition of claim 9 wherein the at least one sunscreen active is a derivative of a compound selected from the group consisting of acetaminosalol; allantoin PABA; 4-aminobenzoic acid; 4-aminobenzoic acid; benzalphthalide; benzophenone-1; benzophenone-2; benzophenone-3; benzophenone-4; benzophenone-5; benzophenone-6; benzophenone-7; benzophenone-8; benzophenone-9; benzophenone-10; benzophenone-11; benzophenone-12; benzotriazolyl dodecyl p-cresol; 3-benzylidene camphor; benzylidenecamphor hydrolyzed collagen sulfonamide; benzylidene camphor sulfonic acid; benzyl salicylate; bisdisulizole disodium; bis(butylbenzoate) diaminotriazine; aminopropyltrisiloxane; bis-ethylhexyloxyphenyl triazine; bornelone; bumetrizole; butyl methoxydibenzoylmethane; calcium cerium oxide; calophyllum inophyllum seed oil; camellia sinensis leaf extract; camphor benzalkonium methosulfate; carotenoids; cerium (3+) carbonate, calcination products with silica; cerium (3+) carbonate, calcination products with silica and talc; cinoxate; cobalt dna; DEA-methoxycinnamate; deschampsia Antarctica leaf extract; diacetylcurcumin; dibenzoxazoyl naphthalene; di-t-butyl hydroxybenzylidene camphor; diethylamino methoxycinnamate; diacetylcurcumin; diethylamino hydroxybenzoyl hexyl benzoate; diethylhexyl butamido triazone; diethylhexyl 2,6-naphthalate; digalloyl trioleate; diisopropylmethyl cinnamate; dimethicone/peg-15 crosspolymer; 1-(3,4-dimethoxyphenyl)-4,4-dimethyl-1,3-pentanedione; di-methoxycinnamidopropyl ethyldimoniuim chloride ether; 4-(dimethylamino)-,3-methylbutyl benzoate; dimethyl PABA ethylcetearyl dimomium tosylate; dimorpholinopyridazinone; diphenyl carbomethoxy acetoxy naphthopyran; diphenylmethyl piperazinylbenzimidazole; disodium bisethylphenyl triaminotriazone; stilbenedisulfonate; disodium distyrylbiphenyl disulfonate; disodium phenyl dibenzimidazole tetrasulfonate; di-t-butyl hydroxybenzylidene camphor; drometrizole; drometrizole trisiloxane; ethyl cinnamate; ethyl dihydroxypropyl PABA; ethyl diisopropyl cinnamate; ethylhexyl bis-isopentylbenzoxazblylphenyl melamine; ethylhexyl dimethoxybenzylidene dioxoimidazolidine propionate; ethylhexyl dimethyl PABA; ethylhexyl ferulate; ethylhexyl methoxycinnamate; 2-ethylhexyl 4-(N-methyl-N-nitrosamino)benzoate; ethylhexyl salicylate; ethylhexyl triazone; ethyl methoxycinnamate; ethylhexyl methoxycrylene; ethylhexyl methoxydibenzoylmethane; ethyl trimethylbenzoyl phenylphosphinate; etocrylene; fluorescent brightener 230; 4-(2-β-glucopyranosiloxy)propoxy-2hydroxybenzophenone; glyceryl ethylhexanoate dimethoxycinnamate; glyceryl ethyklhexanoate dimethoxycinnamate; glyceryl ethylhexanoate dimethoxycinnamate; glyceryl ethylhexanoate dimethoxycinnamate; glyceryl PABA; glycol salicylate; gossypium herbaceum (cotton) seed extract; helianthus annuus (sunflower) seed extract; hexanediol disalicylate; hexyloxy trimethylphenol; homosalate; hydrolyzed aspergillus/ginseng extract ferment; hydrolyzed euglena gracilis extract; hydrolyzed linseed extract; hydrolyzed olive fruit; hydrolyzed wheat bran; hydrolyzed soy extract; 2-hydroxy-1,4-naphthalenedione; hydroxypropyl phenylhydrazinoyl t-butylcarbamate; indocyanine green; isoamyl p-methoxycinnamate; isobutyl phenylhydrazinoyl methanesulfonamide; isopropylbenzyl salicylate; isopropyl dibenzoylmethane; isopropylmethoxy cinnamate; lawsone; limonia acidissima bark extract; limonia acidissima bark powder; Madura aurantica wood extract; maxafil; methoxycinnamidopropyl C18-22 alkyldimonium tosylate; methoxycinnamidopropyl hydroxysultaine; methoxycinnamidopropyl laurdimonium tosylate; methoxycinnamidopropyl polysilsesquioxane; methoxycinnamidopropyl silsesquioxane silicate; methyl acrylate/methylene drometrizole; methacrylate copolymer; menthyl anthranilate; menthyl salicylate; 4-methylbenzylidene camphor; 4-methylbenzylidene camphor sulfonic acid; 5-methykl-2-phenylbenzoxazole; methylene bis-benzotriazolyl tetramethylbutylphenol; momordica cochinchinensis seed aril oil; octocrylene; octrizole; octyl dimethyl 4-aminobenzoic acid; PEG-25 PABA; pentyl 4-(dimethylamino)benzoate; phenylbenzimidazole sulfonic acid; pinus pinaster bark/bud extract; polyacrylamidomethyl benzylidene camphor; polysilicone-15; potassium methoxycinnamate; prinsepia utilis seed extract; red petrolatum; rutinyl succinate; scytonemin; spirulina patensis powder; TEA-salicylate; terephthalylidene dicamphor sulfonic acid; tetrahydrocurcumin diacetate; titanium dioxide; triPABA panthenol; tris-biphenykl triazine; vitis vinifera (grape) seed extract; and zinc cerium oxide.

18. A water-resistant sunscreen composition suitable for topical application comprising:
at least one sunscreen active; and
a water resistance agent comprising hydrolyzed jojoba esters and water.

19. The water-resistant sunscreen composition of claim 18 wherein the hydrolyzed jojoba esters form about 16 wt. % of the sunscreen composition.

20. The water-resistant sunscreen composition of claim 1 wherein the at least one sunscreen active forms about 12 wt. % of the sunscreen composition.
